(19) United States
(12) Patent Application Publication
(10) Pub. No.: US 2025/0232845 A1
(43) Pub. Date: Jul. 17, 2025

(54) MATERIAL SYNTHESIS DEVICE, MATERIAL SYNTHESIS PLATFORM, AND METHOD OF OPERATING MATERIAL SYNTHESIS DEVICE

(71) Applicant: SAMSUNG ELECTRONICS CO., LTD., Suwon-si (KR)

(72) Inventors: Sangyoon Lee, Suwon-si (KR); Dongseon Lee, Suwon-si (KR); Minsu Chang, Suwon-si (KR); Jaejun Chang, Suwon-si (KR); Jaehyung Jang, Suwon-si (KR); Taesin Ha, Suwon-si (KR)

(73) Assignee: SAMSUNG ELECTRONICS CO., LTD., Suwon-si (KR)

(21) Appl. No.: 18/989,808

(22) Filed: Dec. 20, 2024

(30) Foreign Application Priority Data
Jan. 15, 2024 (KR) 10-2024-0006275

(51) Int. Cl. G16C 20/10; G16C 20/70

(52) U.S. Cl. CPC G16C 20/10; G16C 20/70

(57) ABSTRACT

Provided is a material synthesis device, including at least one processor configured to be implemented as a recipe translator configured to, based on analyzing a chemical synthesis recipe, generate a script in the form of a graph representing an antecedent-consequent relationship between target operations corresponding to the chemical synthesis recipe, and a graph scheduler configured to monitor availability of hardware devices of a material synthesis platform, and schedule and execute in parallel commands satisfying the antecedent-consequent relationship in the script based on the monitoring.

### CROSS-REFERENCE TO RELATED APPLICATIONS

This application claims priority to Korean Patent Application No. 10-2024-0006275 filed on Jan. 15, 2024, in the Korean Intellectual Property Office, the disclosure of which is incorporated by reference herein in its entirety.

### BACKGROUND

### 1. Field

Example embodiments of the present disclosure relate to a material synthesis device, a material synthesis platform, and a method of operating the material synthesis device.

### 2. Description of Related Art

Material synthesis is directed to producing target compounds using chemical reactions. A chemical reaction may involve multiple steps of chemical synthesis, and in each step, a chemical analysis may be performed. Chemical synthesis may begin with selecting raw compounds or reagents. A target compound may be obtained through various chemical reactions.

Artificial neural networks (ANNs) may be used to predict chemical reactions and find new compounds. An ANN may receive, as inputs, chemical structures and reaction conditions and predict a new compound and/or a chemical synthesis recipe for producing the new compound. Using such an ANN, material synthesis may find new compounds faster and more efficiently.

### SUMMARY

One or more example embodiments provide a material synthesis device, a material synthesis platform, and a method of operating the material synthesis device.

One or more example embodiments may address at least some of the problems and/or disadvantages described above and/or other disadvantages not described above. In addition, the example embodiments may not be required to overcome the disadvantages described above, and an example embodiment may not overcome any of the problems described above.

According to an aspect of one or more example embodiments, there is provided a material synthesis device, including at least one processor configured to be implemented as a recipe translator configured to, based on analyzing a chemical synthesis recipe, generate a script in the form of a graph representing an antecedent-consequent relationship between target operations corresponding to the chemical synthesis recipe, and a graph scheduler configured to monitor availability of hardware devices of a material synthesis platform, and schedule and execute in parallel commands satisfying the antecedent-consequent relationship in the script based on the monitoring.

The recipe translator may be further configured to generate the script in the form of the graph representing commands configured to perform the target operations in the hardware devices, based on the analyzing of the chemical synthesis recipe and at least one of information with respect to the hardware devices.

The information with respect to the hardware devices may include at least one of a database corresponding to the hardware devices, configuration information of the hardware devices, the antecedent-consequent relationship between the target operations performed on the hardware devices, a movement flow of a robot configured to perform the target operations with respect to a moving target, code corresponding to the hardware devices, and a description language of the hardware devices.

The recipe translator may be further configured to obtain the antecedent-consequent relationship between the target operations based on the chemical synthesis recipe, based on configuration information of the hardware devices.

The recipe translator may be further configured to generate graph blocks, which are respectively a unit of performing the target operations, and structure a synthesis process based on the chemical synthesis recipe by the graph blocks to represent the chemical synthesis recipe as the script.

The script may include node information including information with respect to the hardware devices configured to perform the target operations, detailed information with respect to the target operations, priorities of the target operations based on an importance of the target operations, and a command state configured to perform the target operations, and edge information corresponding to an antecedent-consequent relationship between a preceding node and a following node included in graph blocks.

The command state may include at least one of an idle state, a feasible state, a processing state, a completed state, and an aborted state.

The graph scheduler may include a handler configured to generate and manage a command list including commands satisfying the antecedent-consequent relationship, based on whether execution of preceding nodes is completed in the script, a dispatcher configured to monitor the availability of the hardware devices, and execute at least some of the commands included in the command list by the hardware devices based on the monitoring, and a controller configured to control operations of the handler and the dispatcher, based on a user command from a user.

The handler may be configured to input, into a queue, commands having a command state that is a feasible state among the commands included in the command list, and determine whether the script is completed.

The dispatcher may be configured to score priorities of the commands included in the queue and sort the commands included in the queue in order of highest score, execute the sorted commands based on at least one hardware device that is in an idle state among the hardware devices configured to execute the sorted commands, and change a command state of the sorted commands based on monitoring whether execution of the sorted commands is successful.

The hardware devices may be configured to perform the target operations based on the parallel scheduled script.

The chemical synthesis recipe and the script may be in a JavaScript Object Notation (JSON) file format.

According to another aspect of one or more example embodiments, there is provided a material synthesis platform, including an artificial neural network (ANN) configured to generate a chemical synthesis recipe, a material synthesis device at least one processor configured to generate, based on analyzing the chemical synthesis recipe, a script in the form of a graph representing an antecedent-consequent relationship between target operations corresponding to the chemical synthesis recipe, monitor availability of hardware devices, and schedule, in parallel, commands satisfying the antecedent-consequent relationship in the script based on a result of the monitoring, and at least one hardware module including the hardware devices configured to perform the target operations based on the commands scheduled in parallel.

According to still another aspect of one or more example embodiments, there is provided a method of operating a material synthesis device, the method including analyzing a chemical synthesis recipe, generating, based on analyzing the chemical synthesis recipe, a script in the form of a graph representing an antecedent-consequent relationship between target operations corresponding to the chemical synthesis recipe, monitoring availability of hardware devices of a material synthesis platform, and scheduling and executing, in parallel, commands satisfying an antecedent-consequent relationship in the script based on the monitoring.

The generating of the script may include structuring a synthesis process based on the chemical synthesis recipe by graph blocks, which are a smallest unit of the target operations configured to process a task corresponding to the chemical synthesis recipe, based on the analyzing of the chemical synthesis recipe, allocating hardware resources to the target operations configured to execute the commands configured to perform the graph blocks based on a result of the structuring, and generating the script including the allocated hardware resources and the target operations.

The structuring of the synthesis process may include linking an antecedent-consequent relationship between the graph blocks, determining at least one of detailed commands and parameters configured to perform the graph blocks, assigning identification information to commands including the detailed commands, and linking the identification information by reflecting an antecedent-consequent relationship of the commands to structure the synthesis process based on the chemical synthesis recipe.

The generating of the script may include obtaining the antecedent-consequent relationship between the target operations that execute in parallel a synthesis based on the chemical synthesis recipe, based on a result of analyzing the chemical synthesis recipe and at least one of information about the hardware devices, and generating the script in the form of the graph representing commands for the hardware devices, based on the antecedent-consequent relationship between the target operations.

The scheduling and executing, in parallel, of the commands satisfying the antecedent-consequent relationship in the script may include inputting, into a queue, a command that is in a feasible state among commands stored in a command list including the commands satisfying the antecedent-consequent relationship in the script, and executing commands included in the queue by the hardware devices based on the monitoring of the availability of the hardware devices.

The inputting of the command in the feasible state into the queue may include changing, to a feasible state, a command state of commands whose preceding commands are in a completed state among commands whose command state is an idle state among the commands included in the command list, and inputting, into the queue, the commands whose command state are changed to the feasible state.

The executing of the commands included in the queue may include scoring priorities corresponding to the commands included in the queue and sorting the commands in order of highest score, executing the command based on at least one hardware device that is in an idle state among hardware devices configured to execute the sorted commands, generating a monitoring thread and monitoring whether execution of the command is successful, based on the execution of the command being monitored as being successful, changing a command state of the command to a completed state, and based on the execution of the command being monitored as being unsuccessful, changing the command state of the command to an aborted state.

Additional aspects may be set forth in part in the description which follows and, in part, may be apparent from the description, and/or may be learned by practice of the presented embodiments.

### BRIEF DESCRIPTION OF THE DRAWINGS

The above and/or other aspects, features, and advantages of one or more embodiments may be more apparent from the following description taken in conjunction with the accompanying drawings, in which:

FIG. 1 is a block diagram illustrating a material synthesis device according to an example embodiment;

FIG. 2 is a diagram illustrating operations of a material synthesis device according to an example embodiment;

FIG. 3 is a diagram illustrating a script in the form of a graph according to an example embodiment;

FIG. 4 is a diagram illustrating operations of a recipe translator according to an example embodiment;

FIG. 5 is a diagram illustrating operations of a graph scheduler according to an example embodiment;

FIG. 6 is a diagram illustrating an example graphical user interface (GUI) for graph scheduling according to an example embodiment;

FIG. 7 is a flowchart illustrating a method of operating a material synthesis device according to an example embodiment;

FIG. 8 is a flowchart illustrating an example of generating a script in the form of a graph according to an example embodiment;

FIG. 9 is a flowchart illustrating an example of structuring a synthesis process according to an example embodiment;

FIG. 10 is a flowchart illustrating an example of generating a script in the form of a graph according to an example embodiment;

FIG. 11 is a flowchart illustrating an example of scheduling and executing in parallel commands satisfying an antecedent-consequent relationship in a script according to an example embodiment;

FIG. 12 is a flowchart illustrating an example of inputting, into a queue, commands in a feasible state according to an example embodiment;

FIG. 13 is a flowchart illustrating an example of executing commands included in a queue according to an example embodiment;

FIG. 14 is a flowchart illustrating a method of operating a material synthesis device according to an example embodiment;

FIG. 15 is a flowchart illustrating a method of operating a material synthesis device according to an example embodiment; and

FIG. 16 is a block diagram illustrating a material synthesis platform according to an example embodiment.

### DETAILED DESCRIPTION

The following structural or functional descriptions of example embodiments are provided to merely describe the example embodiments, and the scope of the example embodiments is not limited to the descriptions provided in the disclosure. Various changes and modifications can be made thereto by those of ordinary skill in the art.

Although terms of “first” or “second” are used to explain various components, the components are not limited to the terms. These terms should be used only to distinguish one component from another component. For example, a “first” component may be referred to as a “second” component, or similarly, and the “second” component may be referred to as the “first” component within the scope of the right according to the concept of the present disclosure.

It is to be understood that when a component is referred to as being “connected to” another component, the component can be directly connected or coupled to the other component or intervening components may be present.

As used herein, the singular forms are intended to include the plural forms as well, unless the context clearly indicates otherwise. It should be further understood that the terms “comprises,” “comprising,” “includes,” and/or “including,” when used in this specification, specify the presence of stated features, integers, steps, operations, elements, components or a combination thereof, but do not preclude the presence or addition of one or more other features, integers, steps, operations, elements, components, and/or groups thereof.

Unless otherwise defined herein, all terms used herein including technical or scientific terms have the same meanings as those generally understood by one of ordinary skill in the art. Terms defined in dictionaries generally used should be construed to have meanings matching with contextual meanings in the related art and are not to be construed as an ideal or excessively formal meaning unless otherwise defined herein.

The example embodiments described below may be expanded and applied to various chemical research automation platforms, such as, for example, automated synthesis platforms for new drug development, semiconductors and display materials, and the like.

Hereinafter, the example embodiments are described in detail with reference to the accompanying drawings. When describing the example embodiments with reference to the accompanying drawings, like reference numerals refer to like components and a repeated description related thereto may be omitted.

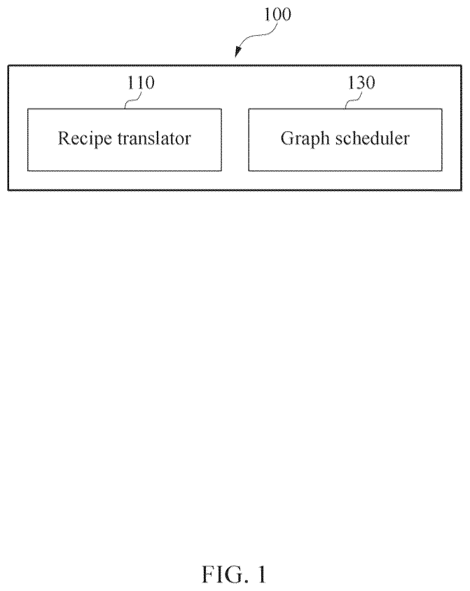

FIG. 1 is a block diagram illustrating a material synthesis device according to an example embodiment. Referring to FIG. 1, a material synthesis device 100 according to an example embodiment may include a recipe translator 110 and a graph scheduler 130.

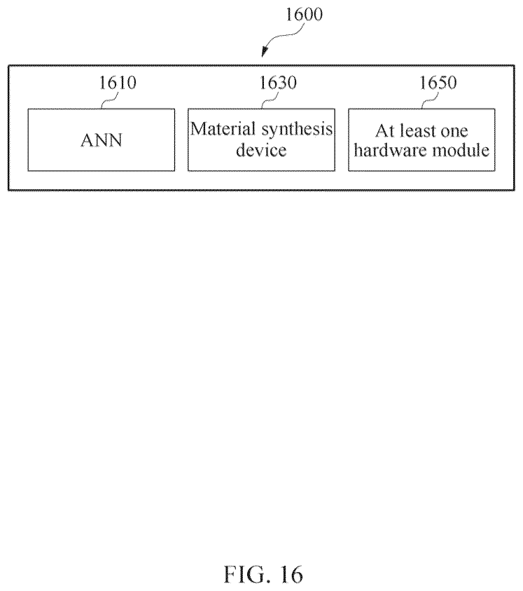

The recipe translator 110 may generate a script in the form of a graph that represents an antecedent-consequent relationship between target operations corresponding to a chemical synthesis recipe, based on an analysis result obtained by analyzing the chemical synthesis recipe. The script in the form of a graph may also be referred to herein as a graph script for ease of description. In this case, the chemical synthesis recipe may be, but is not necessarily limited to, an experiment recipe for chemical synthesis generated or predicted by an artificial neural network (ANN) (e.g., an ANN 1610 in FIG. 16). The chemical synthesis recipe and/or script may have, for example, but is not necessarily limited to, a JavaScript Object Notation (JSON) file format. JSON may refer to an open standard format that uses human-readable text to convey data objects consisting of, for example, attribute-value pairs, array data types, other serializable values, or key-value pairs. JSON may represent data structured by the JavaScript object syntax. For example, JSON may be used to transfer data in web applications.

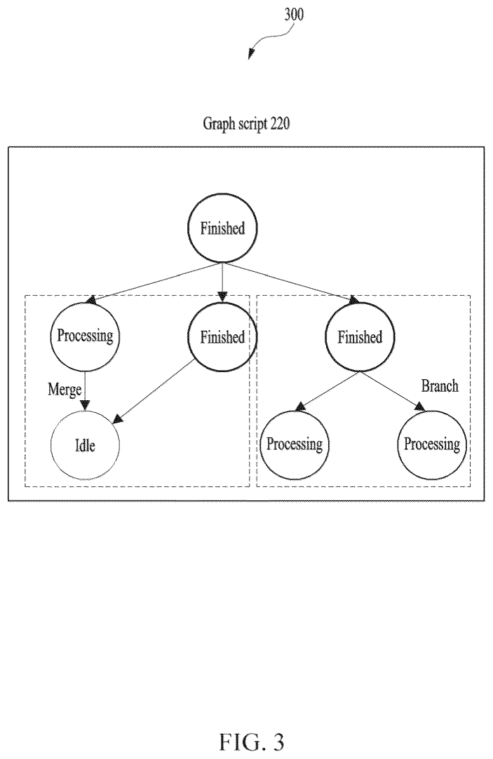

The graph script may correspond to a command script in the form of a graph representing feasible target operations in parallel. The graph script will be described in more detail below with reference to FIG. 3.

The recipe translator 110 may generate a script that represents, in the form of a graph, commands for performing target operations on hardware devices, based on an analysis result of the chemical synthesis recipe and at least one of information about the hardware devices. The information about the hardware devices may include, for example, at least one of a database (DB) corresponding to the hardware devices, configuration information of the hardware devices, an antecedent-consequent relationship between the target operations performed on the hardware devices, a movement flow of a robot for performing the target operations with respect to a moving target, code corresponding to the hardware devices, and a description language of the hardware devices. The description language of the hardware devices may be, for example, but not necessarily limited to, a programming language such as Python, Ruby, Scala, Java, C++, C#, and the like, or a markup language such as HyperText Markup Language (HTML), Extensible Markup Language (XML), JSON, Cascading Style Sheets (CSS), JavaServer Pages (JSP), and the like.

The recipe translator 110 may derive and obtain the antecedent-consequent relationship between the target operations according to the chemical synthesis recipe, based on the configuration information of the hardware devices.

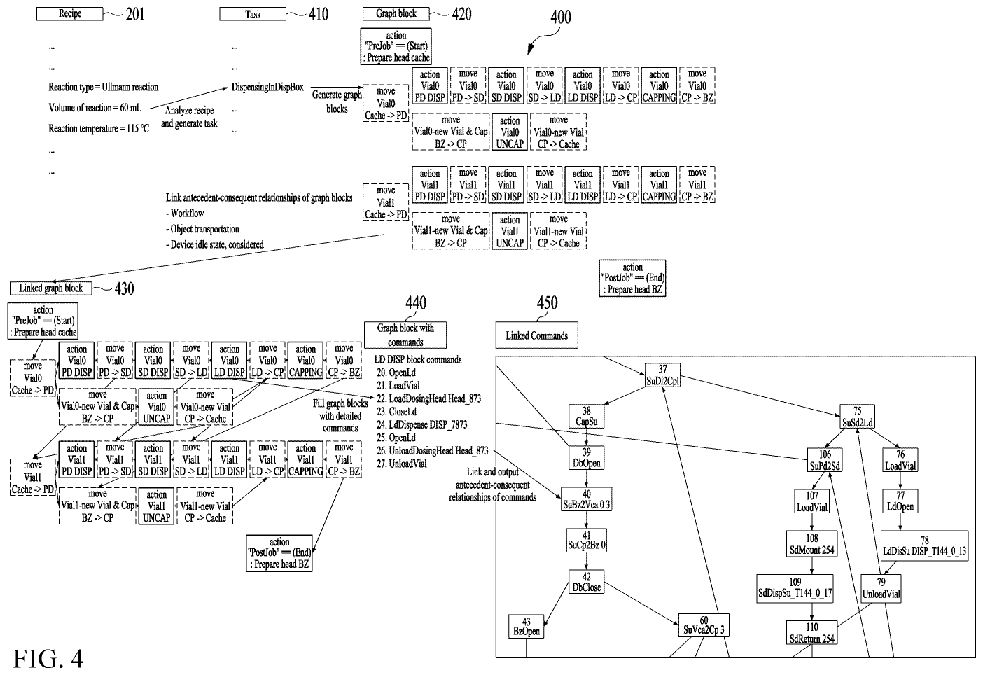

The recipe translator 110 may represent the chemical synthesis recipe as the script by generating graph blocks, which are a unit of performing the target operations, and structuring a synthesis process according to the chemical synthesis recipe by the graph blocks. A process in which the recipe translator 110 structures a synthesis process by graph blocks and represents it in the form of a graph will be described in more detail below with reference to FIG. 4.

The graph scheduler 130 may monitor the availability of hardware devices of a material synthesis platform and, based on a monitoring result obtained by the monitoring, schedule and execute in parallel commands that satisfy an antecedent-consequent relationship in the script.

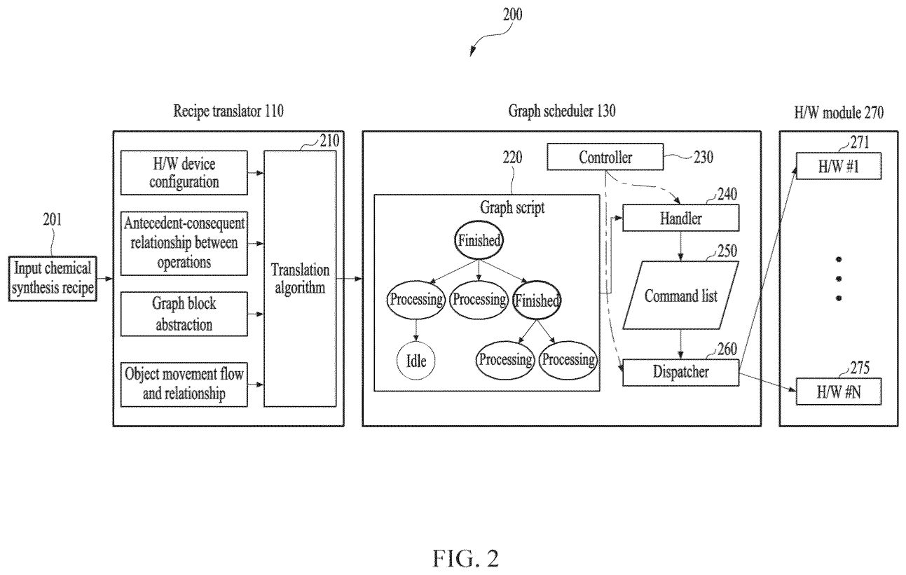

The graph scheduler 130 may include, for example, a handler, a dispatcher, and a controller. The handler may generate and manage a command list including the commands that satisfy the antecedent-consequent relationship based on whether the execution of preceding nodes in the script has been completed. The handler may also update a state of the script and generate and manage a command list including commands corresponding to the updated state of the script. The handler may input, into a queue, commands whose command state is a feasible state among the commands included in the command list, and check whether the script is completed. The command state will be described in more detail below with reference to FIG. 2. The dispatcher may monitor the availability of the hardware devices and, based on a monitoring result, execute at least some of the commands included in the command list by the hardware devices. In this case, the hardware devices may perform the target operations according to the parallel scheduled script.

The dispatcher may score priorities of the commands included in the queue and sort the commands in the queue in order of highest score. The dispatcher may execute the sorted commands using at least one hardware device that is in an idle state among hardware devices on which the sorted commands are to be executed. The dispatcher may change a command state of the sorted commands based on a monitoring result of whether the execution of the sorted commands is successful. The controller may control the operations of the handler and the dispatcher based on a user command from a user, for example.

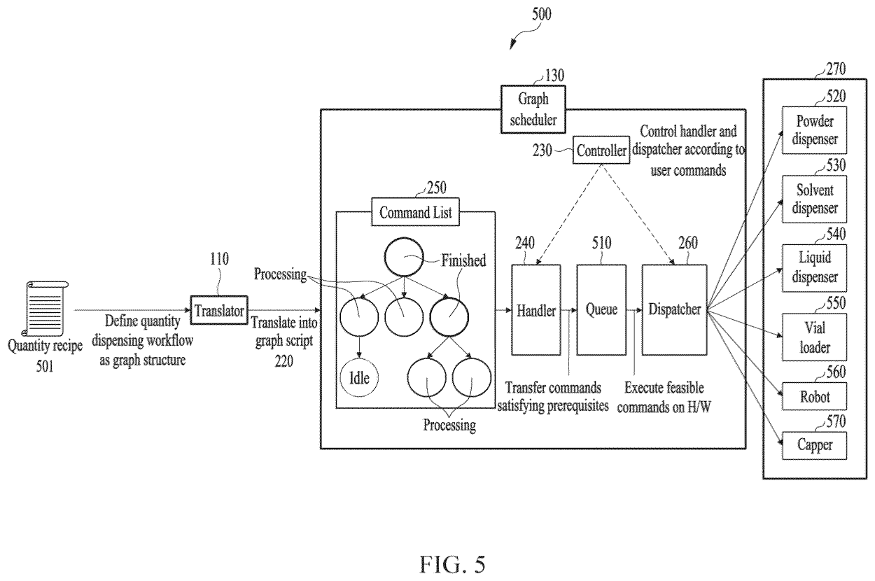

The structure and operations of the graph scheduler 130 will be described in more detail below with reference to FIG. 5

For example, when a file including a chemical synthesis recipe in the JSON format is input, the material synthesis device 100 may output a graph script including hardware commands in the JSON format.

FIG. 2 is a diagram illustrating operations of a material synthesis device according to an example embodiment. Referring to FIG. 2, diagram 200 shows operations performed between the material synthesis device including the recipe translator 110 and the graph scheduler 130 and hardware devices (e.g., a first hardware device 271, . . . , and an Nth hardware device, for example, fifth hardware device 275) of a hardware module 270 of a material synthesis platform when a chemical synthesis recipe 201 is input to the material synthesis device.

The recipe translator 110 may receive, as an input, the chemical synthesis recipe 201 and generate a script 220 in which commands for the hardware devices 271, 272, 273, 274, and 275 are represented in the form of a graph. In this case, the chemical synthesis recipe 201 may correspond to, but is not necessarily limited to, a chemical synthesis recipe according to a synthesis pathway predicted by an ANN based on an input such as a reaction condition.

The recipe translator 110 may analyze a chemical synthesis process according to the chemical synthesis recipe 201 based on unique characteristics of the material synthesis platform, and derive and obtain an antecedent-consequent relationship between commands based on constraints on the hardware devices 271 to 275 of the hardware module 270. In this case, the antecedent-consequent relationship between the commands may have the form of a complex graph.

The recipe translator 110 may generate the script 220 that represents, in the form of a graph, the commands for the hardware devices 271 to 275 based on an analysis result of the chemical synthesis recipe 201 and at least one of information about the hardware devices 271 to 275. The recipe translator 110 may generate the script 220 that represents, in the form of a graph, the commands for the hardware devices 271 to 275 based on the analysis result. For example, the recipe translator 110 may derive and obtain an antecedent-consequent relationship between target operations that may execute a synthesis operation(s) according to the chemical synthesis recipe 201 based on configuration information of the hardware devices 271 to 275.

The recipe translator 110 may generate graph blocks for processing a task. The graph blocks may be executed sequentially. The recipe translator 110 may abstract operations (e.g., store, transport, dispense, react, aliquot, analyze, etc.) in the chemical synthesis recipe 201 into the graph blocks by each reusable unit of repetitive operations. As used herein, abstracting operations in a chemical synthesis recipe into graph blocks may include chemical synthesis operations according to the chemical synthesis recipe as graph blocks, which are the smallest unit of the operations. The recipe translator 110 may structure the chemical synthesis process by the graph blocks to represent the chemical synthesis recipe 201 as the script 220 in the form of a graph. The script 220 in the form of a graph may simply be referred to herein as a graph script 220.

The recipe translator 110 may analyze the chemical synthesis recipe 201, which is information independent of the hardware devices 271 to 275, and the information about the hardware devices 271 to 275, which is information dependent on the hardware devices 271 to 275. The information about the hardware devices 271 to 275 may include, for example, at least one of a DB corresponding to the hardware devices 271 to 275, configuration information of the hardware devices 271 to 275, an antecedent-consequent relationship between target operations performed on the hardware devices 271 to 275, a movement flow of a robot for performing the target operations with respect to a moving target, code corresponding to the hardware devices 271 to 275, and a description language of the hardware devices 271 to 275.

Since chemical information in the chemical synthesis recipe 201 may be variable depending on a chemical synthesis process, the recipe translator 110 may structure the chemical synthesis process by a translation algorithm 210 to represent the chemical synthesis recipe 201 in the form of a graph. The recipe translator 110 may translate the chemical synthesis recipe 201 into the graph script 220 by the translation algorithm 210. The translation algorithm 210 may correspond to an algorithm that translates the chemical synthesis recipe 201 that is received as an input into the graph script 220 that is in the form of a graph. For example, the translation algorithm 210 may represent the merging and branching in the graph script 220 in a unit of graph blocks rather than in a unit of commands. The transformation algorithm 210 may be provided in various forms reflecting a material synthesis platform, a hardware module, and a priority from the user. The translation algorithm 210 may generate the graph script 220, for example, in a JSON file format.

The graph script 220 generated by the translation algorithm 210 may be stored, in the JSON file format, in a file storage in an operating system (OS) such as the material synthesis platform.

For example, in a case where the chemical synthesis recipe 201 for manufacturing six vials is input, the recipe translator 110 may define, as a graph block X, operations that are executable in sequential order among unit operations for one vial. In this case, when translating operations of manufacturing the six vials into the script 220, the recipe translator 110 may reuse the previously defined graph block X. The recipe translator 110 may determine a connecting relationship (e.g., a link) between graph blocks based on an antecedent-consequent relationship between the operations. The recipe translator 110 may generate the graph script 220 by reflecting therein a movement flow of objects such as the vials. To enable the material synthesis platform to more flexibly perform a changing chemical synthesis process, the recipe translator 110 may generate the script 220 by reflecting therein all information that may change in each chemical synthesis recipe, such as, for example, the number of vials, the type of reagents dispensed, and the like.

The graph scheduler 130 may receive the graph script 220 as an input, store it in an internal memory, and prepare to execute the operations according to the script 220.

For example, the graph scheduler 130 may receive the graph script 220 and control the hardware devices (e.g., the first hardware device H/W #1 271, . . . , and the Nth hardware device H/W #N, for example, fifth hardware device 275) in the hardware module 270 to execute the target operations in parallel.

The graph scheduler 130 may be required to flexibly respond to uncertainties in an execution time of each operation according to the chemical synthesis recipe 201 while, simultaneously, monitoring an accurate execution state of commands according to the graph script 220 and a state of the hardware devices 271 to 275.

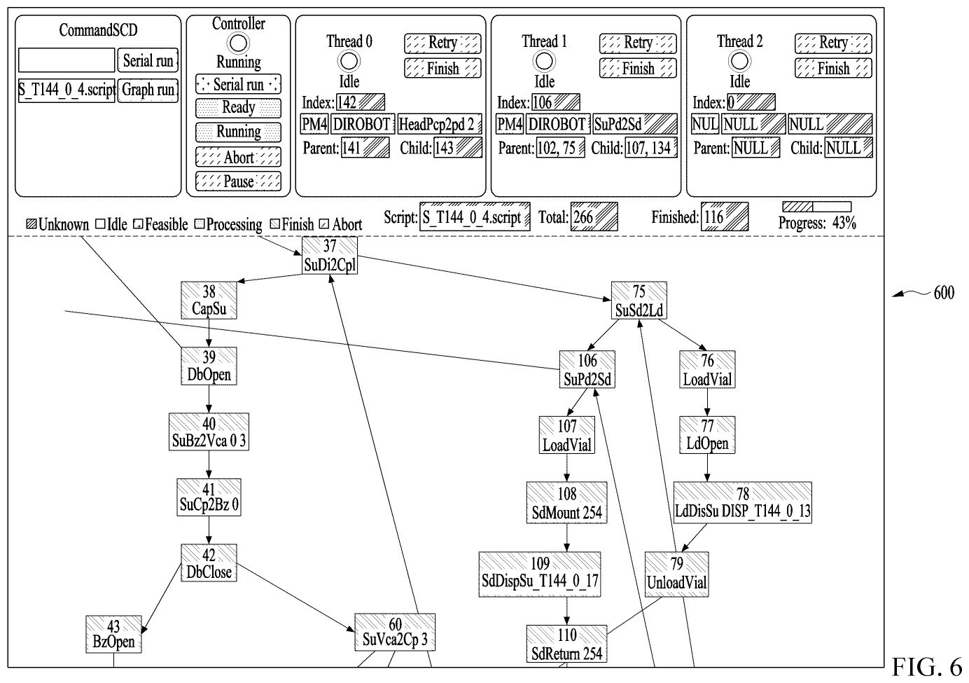

During parallel scheduling, multiple operations may be performed simultaneously on various hardware devices (e.g., 271 to 275). Accordingly, the material synthesis device may provide a graphical user interface (GUI) for graph scheduling, or a graph scheduling GUI (e.g., a GUI 600 in FIG. 6), such that the user identifies a progress of the chemical synthesis process at a glance. In the event of an exceptional situation, the user may interrupt and/or recover the chemical synthesis process by manual control through the graph scheduling GUI 600. The graph scheduler 130 may also use detailed algorithms to perform scheduling that reflects priorities defined by the user.

The graph scheduler 130 may include, for example, a controller 230, a handler 240, and a dispatcher 260.

The controller 230 may control the operations of the handler 240 and the dispatcher 260 based on user commands from the user.

The handler 240 may manage commands in the graph script 220, and may traverse the script 220 to check whether the execution of preceding nodes has been completed and store commands satisfying an antecedent-consequent relationship in a command list 250. The commands stored in the command list 250 may be commands that satisfy an antecedent-consequent relationship in a graph and may be executed directly by the dispatcher 260 when the hardware devices 271 to 275 are in an idle state (also available state).

The handler 240 may check whether the execution of the script 220 is entirely completed.

The handler 240 may update a state of the graph script 220. The handler 240 may generate and/or manage the command list 250 including commands corresponding to the updated state of the script 220.

The dispatcher 260 may monitor the availability of the hardware devices 271 to 275. The dispatcher 260 may traverse or refer to the command list 250 to execute at least some of the commands included in the command list 250 by the hardware devices 271 to 275, based on a monitoring result of the hardware devices 271 to 275.

While monitoring the availability of the hardware devices 271 to 275, when the hardware devices 271 to 275 are monitored as being available, the dispatcher 260 may execute the commands included in the command list 250 on the available hardware devices 271 to 275. The dispatcher 260 may also monitor whether the execution of a command is completed on each of the hardware devices 271 to 275. The dispatcher 260 may check whether the execution of a command has been completed on each of the hardware devices 271 to 275 and may record, in node information of the script 220, whether the execution is completed. For example, when at least one of the hardware devices 271 to 275 is available as a monitoring result, the dispatcher 260 may execute a command included in the command list 250 by the available hardware device. The dispatcher 260 may perform operations, such as, for example, managing resources, allocating operations, and monitoring results.

In an example embodiment, the traversal of the script 220 by the handler 240 and the traversal of the command list 250 by the dispatcher 260 may be performed by a search algorithm specific to the hardware module 270. The material synthesis device may coordinate the efficiency of its operations by a priority-based search algorithm specific to the hardware module 270. The search algorithm may include, for example, an algorithm based on real-time monitoring and dispatching. The search algorithm may flexibly respond to uncertainties of execution times of the hardware devices 271 to 275, which may be suitable for a material synthesis platform where different chemical synthesis recipes have different execution times.

The material synthesis device according to an example embodiment may analyze a process according to the chemical synthesis recipe 201 through the recipe translator 110 and the graph scheduler 130 to translate the chemical synthesis recipe 201 into commands suitable for the hardware devices 271 to 275 included in the hardware module 270.

The hardware module 270 may include, for example, but is not necessarily limited to, any one or a combination of a storage device, a carrier, a dispenser, a reactor, a collector, and an analyzer. The storage device may be a device for storing reagents in a constant environment. The storage device may be, for example, a refrigerator, a warmer, or a vacuum chamber. The carrier may be a device for moving or transporting reagents or tools to specific locations. The carrier may be, for example, a transfer robot, a lift, or a conveyor belt. The reactor may include a reaction vessel in which a chemical reaction occurs, and may further include a heater or pump for regulating a temperature or gas composition ratio in the reaction vessel. The dispenser may be used to inject reagents into the reaction vessel. The collector may be used to collect a sample from the reaction vessel. The dispenser and the collector may be, for example, a syringe, pipette, burette, or dropper. The analyzer may be used to analyze the sample and, if necessary, may perform pretreatment on the sample for the analysis. The pretreatment may refer to any treatment performed on a sample before the analysis of the sample to ensure an accurate analysis of the sample. The pretreatment may include, for example, but is not necessarily limited to, precipitation, filtration, distillation, and extraction. The analyzer may be, for example, a scale, a chromatographer, or a spectrometer.

The material synthesis device may support the parallel scheduling that reflects the physical constraints of the hardware module 270, and it may thus be applicable to various forms of hardware modules 270 and may abstract the hardware module 270 to support interworking with other hardware modules.

FIG. 3 is a diagram illustrating a graph script according to an example embodiment. Referring to FIG. 3, diagram 300 shows a graph script 220 according to an example embodiment.

The material synthesis device may structure each task as the graph script 220 by defining each command as a node and an antecedent-consequent relationship between commands as an edge.

The graph script 220 may include node information and edge information. The node information may include, for example, information about hardware devices performing target operations, detailed information about the target operations, priorities of the target operations based on their importance, and a command state for performing the target operations. The command state may include, for example, at least one of an idle state, a feasible state, a processing state, a completed state, and an aborted state. The edge information may indicate an antecedent-consequent relationship between a preceding node(s) and a following node(s) in graph blocks included in the graph script 220. Here, the antecedent-consequent relationship between the preceding node(s) and the following node(s) may refer to a relationship in which, after the execution of a preceding node is completed, a next node (e.g., a following node) connected by an edge is performed.

For example, when commands 2 and 3 are executable at the same time after command 1 is executed, commands 2 and 3 may be children of command 1, and at the same time, command 1 may be a parent of commands 2 and 3. For example, the execution of a parent (e.g., command 1) may need to be completed first to enable its children (e.g., commands 2 and 3) to be executable.

As described above, a case where two or more commands executable in parallel are separated from a single command may be defined as branching or branch. In addition, a case where, for a command, there are two or more preceding commands command may be defined as merging or merge. When there is no branching and merging, commands corresponding nodes in the graph script 220 may be executed sequentially. The antecedent-consequent relationship between commands may be derived based on both the chemical synthesis process and the constraints of hardware devices.

FIG. 4 is a diagram illustrating operations of a recipe translator according to an example embodiment. Referring to FIG. 4, diagram 400 shows a process in which a recipe translator (e.g., the recipe translator 110 in FIG. 1) according to an example embodiment receives, as an input, a chemical synthesis recipe 201 and information about hardware devices and generates a script in the form of a graph (also a graph script herein).

The recipe translator may output, as a graph script, execution commands suitable for hardware devices of a material synthesis platform in consideration of information about the hardware devices, based on a hardware device-independent file (e.g., the chemical synthesis recipe 201) that expresses the intent of an experiment.

For example, when the chemical synthesis recipe 201 for checking a reaction to a 60 milliliters (mL) compound is input, the recipe translator may generate a command to divide the 60 mL compound into six vials to fit the size (e.g., 10 mL) of a reactor for the reaction, based on information about hardware devices. According to another example embodiment, for example, the chemical synthesis recipe 201 may state that 10 mmol (1 g) of material A (100 g/mol) is used for reaction Y, but the material A provided in a hardware device may be a product with a 97% purity. In this case, the recipe translator may generate, based on the purity of the material A, a command to dispense an amount (e.g., 1.03 g) according to the purity, rather than an amount (e.g., 10 mmol (1 g)) recorded in the chemical synthesis recipe 201.

In this example, the recipe translator may receive the chemical synthesis recipe 201. The chemical synthesis recipe 201 may include information about a chemical reaction including, such as, for example, a reaction type (e.g., Ullmann reaction), a reaction volume (e.g., 60 mL), and a reaction temperature (e.g., 115° C.), along with information such as a chemical reaction pathway and dispensing information. In this example, the dispensing information may include, for example, information such as solid reagents A, B, and C, solvent D, and the respective dispensing amounts.

The recipe translator may generate a task 410 based on an analysis result of the chemical synthesis recipe 201. The task 410 may correspond to, but is not necessarily limited thereto, a set of operations performed by hardware devices of each hardware module. For example, the task 410 may include a dispensing task for a dispensing box (e.g., dispensing in DispBox).

The recipe translator may generate graph blocks 420 for processing the task 410. The graph blocks 420 may be executed sequentially. The recipe translator may abstract operations in the chemical synthesis recipe 201 into the graph blocks 420 as reusable units for repetitive operations. The graph blocks 420 may include, for example, but are not necessarily limited to, move blocks and action blocks.

The recipe translator may derive and obtain an antecedent-consequent relationship between operations that may execute the operations of the chemical synthesis recipe 201, based on configuration information of the hardware devices.

The recipe translator may generate a linked graph block 430 by linking antecedent-consequent relationships of the graph blocks 420 in such a parent-child relationship. The recipe translator may generate the linked graph block 430 by linking the antecedent-consequent relationships of the graph blocks 420 based on, for example, a workflow, an object transportation by a robot, and an idle state of a device. The recipe translator may generate a graph block 440 with commands that includes commands by filling each linked graph block 430 with detailed commands and/or parameters for performing each of the graph blocks 420.

The recipe translator may generate and output a graph script by linked commands 450 that link antecedent-consequent relationships of commands by assigning identification information to commands including the detailed commands. In this case, the recipe translator may link the identification information by reflecting the antecedent-consequent relationships of the commands and allocating hardware resources to target operations for executing commands to generate the graph script including the allocated hardware resources and target operations.

FIG. 5 is a diagram illustrating operations of a graph scheduler according to an example embodiment. Referring to FIG. 5, diagram 500 shows a process in which the graph scheduler 130 according to an example embodiment receives, as an input, a graph script 220 from the recipe translator 110 and executes a task (e.g., a chemical synthesis) by the hardware module 270.

The hardware module 270 may correspond to a set of devices that is not dependent on a chemical synthesis recipe and may be provided to perform a unit function. The types of chemical synthesis recipes that may be processed by the hardware module 270 are not limited, and a variety of chemical synthesis recipes may be executed on the hardware module 270 as long as they are translated into appropriate hardware commands by the recipe translator 110.

In a case where a material synthesis platform supports two types of experiments, for example, an optimization experiment and an enhancement experiment (also referred to herein as a quantity experiment), the recipe translator 110 may translate, into a graph script, a chemical synthesis recipe (e.g., a quantity recipe 501) for each of the optimization experiment and the enhancement experiment, and the graph scheduler 130 may execute chemical synthesis operations by the hardware module 270 according to the graph script.

The hardware module 270 may be, for example, but is not necessarily limited to, a dispensing module configured to perform automated dispensing of reagents on the material synthesis platform. In this case, hardware devices included in the hardware module 270 may include, for example, a powder dispenser 520, a solvent dispenser 530, a liquid dispenser 540, a vial loader 550, a robot 560, and a capper 570. In the hardware module 270, there is one dispenser for each of the reagents according to characteristics of the reagents, and thus the material synthesis device may dispense three different chemical synthesis recipes simultaneously. Using typical sequential scheduling, when one dispenser is dispensing, the remaining dispensers are unavailable, which may significantly degrade the usability of a dispensing module. However, according to an example embodiment, the graph scheduler 130 may analyze a relationship between the chemical synthesis recipes and the constraints (e.g., an antecedent-consequent relationship of processes, etc.) of the dispensing module and derive and obtain an antecedent-consequent relationship in a graphs to process the multiple chemical synthesis recipes, thereby improving the usability of the material synthesis device.

For example, in a case where the quantity recipe 501 is given, the recipe translator 110 may generate a graph script 220 that may execute hardware devices 520, 530, 540, 550, 560, and 570 of the hardware module 270 based on information included in the quantity recipe 501. The recipe translator 110 may translate a workflow for quantity dispensing (or incremental dispensing) into the graph script 220 that defines the workflow in the form of a graph. In this case, a chemical synthesis recipe including the quantity recipe 501 may be input to the recipe translator 110 in the form of a JSON file. The chemical synthesis recipe may include dispensing-related information such as chemical reaction pathways, chemical reaction conditions, and the like. The dispensing-related information may include, for example, information such as solid reagents A, B, and C, solvent D, and their respective dispensing amounts. The quantity recipe 501 may be, for example, a chemical synthesis recipe synthesized by an ANN.

The graph scheduler 130 may monitor the availability of the hardware devices 520, 530, 540, 550, 560, and 570 included in the hardware module 270 of the material synthesis platform and, based on a monitoring result, schedule in parallel the graph script 220 to execute a chemical synthesis.

The graph scheduler 130 may include, for example, a controller 230, a handler 240, and a dispatcher 260.

The controller 230 may control the operations of the handler 240 and the dispatcher 260 in response to user commands from a user.

The handler 240 may update a state of the graph script 220. The handler 240 may generate and/or manage a command list 250 including commands corresponding to the graph script 220 and/or the updated script state. When the graph script 220 is input, the handler 240 may store and manage the commands corresponding to the graph script 220 and/or the updated script state in the command list 250, for example, in the form of a C++ vector data structure.

The handler 240 may store, in a queue 510, commands that satisfy prerequisites (e.g., a condition that the execution of preceding commands is completed, a condition that a hardware device is available, a condition that the execution is in a completed state) among commands included in the command list 250, and may then check whether the script 220 is completed entirely. For example, the handler 240 may search for a command whose preceding commands are all in a completed state among commands whose command state is an idle state in the command list 250, and change a command state of the command to a feasible state. In this case, the handler 240 may place commands in the feasible state among the commands included in the command list 250 into the queue 510 that is checked by the dispatcher 260 to execute the command. When all the commands stored in the queue 510 are in the completed state, the execution of the script 220 may be completed successfully.

The dispatcher 260 may execute the commands stored in the queue 510 while monitoring the queue 510 storing the feasible commands and the state of the hardware devices 520, 530, 540, 550, 560, and 570.

The state of the hardware devices 520, 530, 540, 550, 560, and 570 may be represented by, for example, but is not necessarily limited to, an idle state (or available state), a processing state, and an aborted state.

The dispatcher 260 may sort the commands included in the queue 510. The dispatcher 260 may score the priorities of the commands included in the queue 510 and sort the commands in order of highest score.

The dispatcher 260 may monitor the availability of the hardware devices 520, 530, 540, 550, 560, and 570 on which the sorted commands are to be executed. The dispatcher 260 may execute at least some of the commands included in the command list 250 by the hardware devices 520, 530, 540, 550, 560, and 570 based on a monitoring result of the hardware devices 520, 530, 540, 550, 560, and 570. In this case, the hardware devices 520, 530, 540, 550, 560, and 570 may perform target operations according to the parallel scheduled script.

The dispatcher 260 may execute a command using at least one hardware device that is in the idle state among the hardware devices 520, 530, 540, 550, 560, and 570. For example, when hardware devices s and t on which command A among commands having a higher priority is to be executed are in the idle state, the dispatcher 260 may execute the command A by the hardware devices s and t that are in the idle state. In this case, the command A may be invoked by an application programming interface (API) of the graph scheduler 130, and may thus be executed in the same form independently on the hardware device(s) (e.g., s and t).

After the execution of the command, the dispatcher 260 may generate a monitoring thread to determine whether the execution of the command is successful or unsuccessful. The dispatcher 260 may change a command state of the command based on a monitoring result of whether the command is executed successfully. For example, when the execution of the command is successful, the dispatcher 260 may change the command state to the completed state and transfer the changed command state to the handler 240. When the execution of the command is unsuccessful, the dispatcher 260 may change the command state of the command to the aborted state and notify an operator that the execution has failed.

FIG. 6 is a diagram illustrating an example GUI for graph scheduling according to an example embodiment. Referring to FIG. 6, an example screen of a graph scheduling GUI 600 provided to a user is shown.

The material synthesis device may provide the graph scheduling GUI 600 based on a user interface (UI) such that the user may identify the progress of a chemical synthesis process at a glance.

In this case, the UI may correspond to a means for inputting a synthesis method used to synthesize a target product. The UI may be, for example, but is not limited to, a key pad, a dome switch, a touch pad (contact capacitive, pressure sensitive resistive, infrared sensing, surface ultrasonic conduction, integral tension measurement, piezo effect, etc.), a jog wheel, a jog switch, and the like. The UI may include a display means, and the user may monitor a synthesis result through the display means.

In the event of an exceptional situation, the user may interrupt and/or recover a chemical synthesis process by manual control through the graph scheduling GUI 600. The material synthesis device may also use a detailed algorithm to perform scheduling by applying priorities defined (or set) by the user through the graph scheduling GUI 600, and to control the operations of hardware modules.

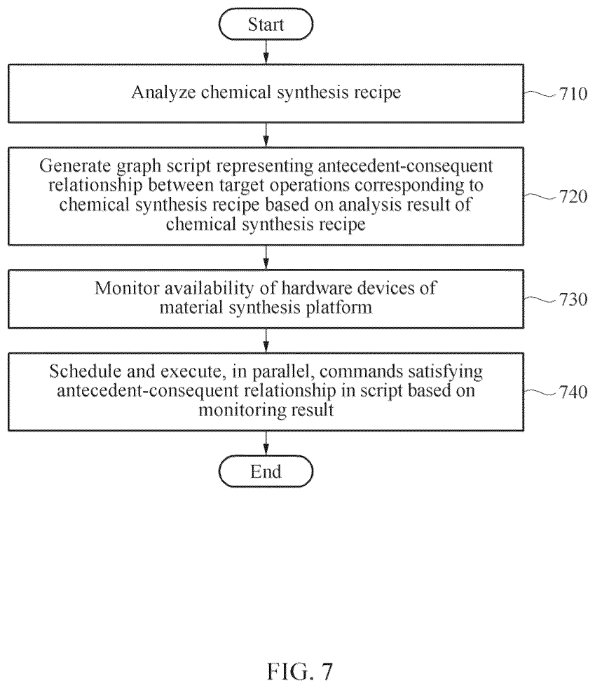

FIG. 7 is a flowchart illustrating a method of operating a material synthesis device according to an example embodiment. In the embodiments described with reference to FIG. 7 and subsequent drawings, steps to be described below with reference to FIG. 7 and subsequent drawings may be performed in sequential order but not be performed necessarily in sequential order. For example, the order of the steps may be changed and at least two of the steps may be performed in parallel, or one step may be divided or an additional step may be added.

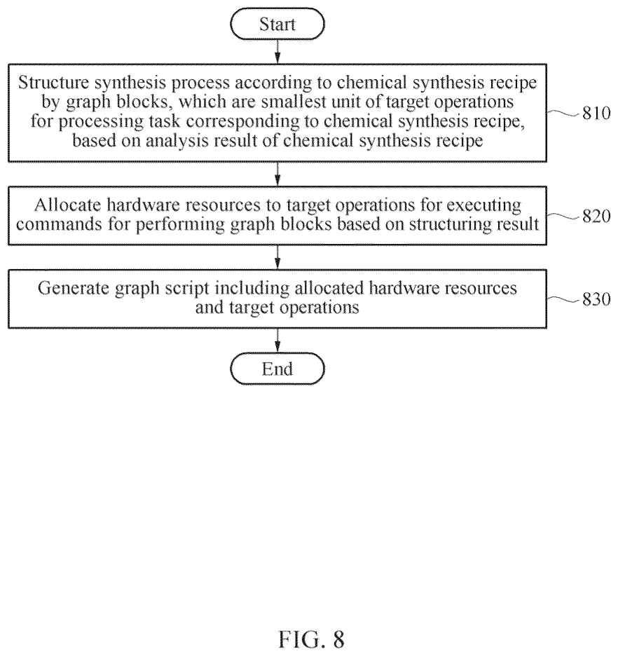

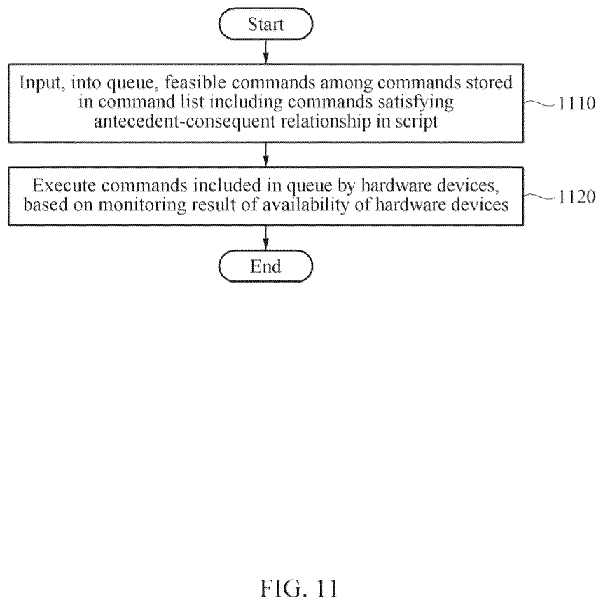

Referring to FIG. 7, the material synthesis device may execute commands that are executable by hardware devices in steps 710 to 740 described below.
In step 710, the material synthesis device may analyze a chemical synthesis recipe.
In step 720, the material synthesis device may generate a graph script representing an antecedent-consequent relationship between target operations corresponding to the chemical synthesis recipe based on an analysis result obtained by analyzing the chemical synthesis recipe in step 710. Step 710 in which the material synthesis device generates a graph script will be described in more detail below with reference to FIGS. 8 to 10.
In step 730, the material synthesis device may monitor the availability of hardware devices of a material synthesis platform.
In step 740, the material synthesis device may schedule and execute in parallel commands that satisfy an antecedent-consequent relationship in the script based on a monitoring result obtained in step 730. Step 730 in which the material synthesis device schedules and executes in parallel commands will be described in more detail below with reference to FIGS. 11 to 13.

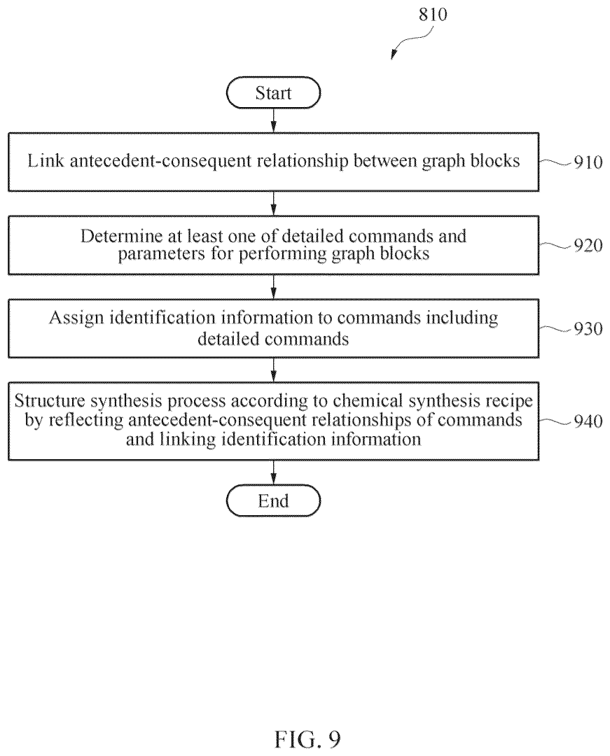

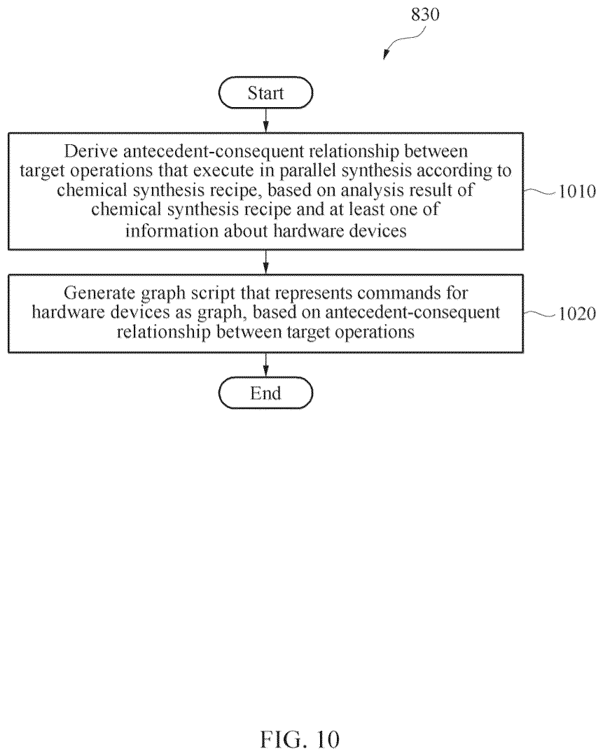

FIG. 8 is a flowchart illustrating an example of generating a graph script according to an example embodiment. Referring to FIG. 8, the material synthesis device may generate a graph script in step 720 through steps 810 to 830 described below.
In step 810, the material synthesis device may structure a synthesis process according to a chemical synthesis recipe by graph blocks, which are the smallest unit of target operations for processing a task corresponding to the chemical synthesis recipe, based on an analysis result of the chemical synthesis recipe. Step 810 in which the material synthesis device structures a synthesis process will be described in more detail below with reference to FIG. 9.
In step 820, the material synthesis device may allocate hardware resources to target operations for executing commands for performing the graph blocks based on a structuring result obtained in step 810.
In step 830, the material synthesis device may generate a graph script including the hardware resources and target operations allocated in step 830. Step 830 in which the material synthesis device generates a graph script will be described in more detail below with reference to FIG. 10.

FIG. 9 is a flowchart illustrating an example of structuring a synthesis process according to an example embodiment. Referring to FIG. 9, the material synthesis device according to an example embodiment may structure a synthesis process in step 810 through steps 910 to 940 described below.
In step 910, the material synthesis device may link an antecedent-consequent relationship between graph blocks.
In step 920, the material synthesis device may determine at least one of detailed commands and parameters for performing the graph blocks linked in step 910.
In step 930, the material synthesis device may assign identification information to commands including the detailed commands.
In step 940, the material synthesis device may structure a synthesis process according to a chemical synthesis recipe by reflecting an antecedent-consequent relationship of the commands and linking the identification information assigned in step 930.

FIG. 10 is a flowchart illustrating an example of generating a graph script according to an example embodiment. Referring to FIG. 10, the material synthesis device according to an example embodiment may generate a graph script in step 830 through steps 1010 and 1020 described below.
In step 1010, the material synthesis device may derive and obtain an antecedent-consequent relationship between target operations that may be executed in parallel for a synthesis according to a chemical synthesis recipe, based on an analysis result of the chemical synthesis recipe and at least one of information about hardware devices.
In step 1020, the material synthesis device may generate a graph script that represents commands for the hardware devices in the form of a graph, based on the antecedent-consequent relationship between the target operations derived in step 1010.

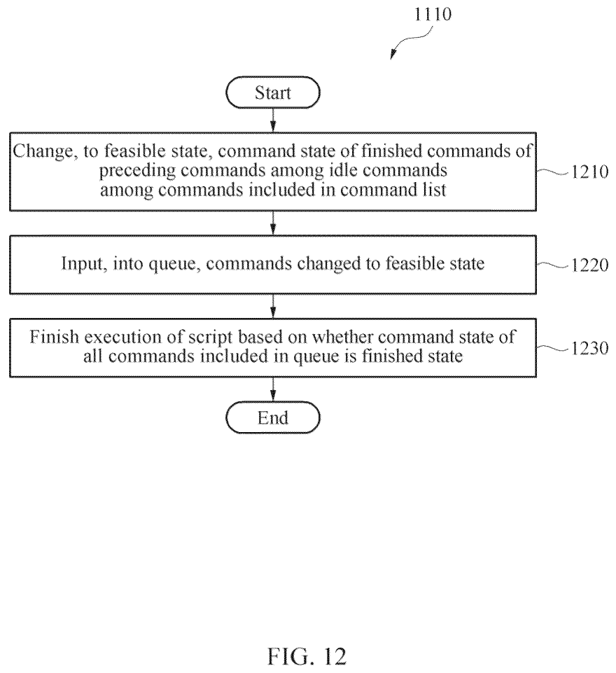

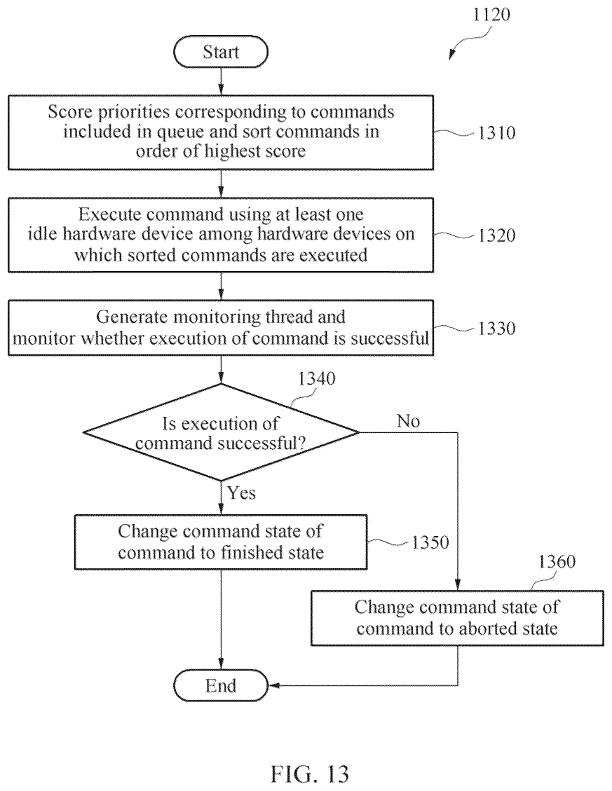

FIG. 11 is a flowchart illustrating an example of scheduling and executing, in parallel, commands satisfying an antecedent-consequent relationship in a script according to an example embodiment. Referring to FIG. 11, the material synthesis device according to an example embodiment may schedule and execute in parallel commands in step 740 through steps 1110 and 1120 described below.
In step 1110, the material synthesis device may input, into a queue, commands that are in a feasible state among commands stored in a command list including commands satisfying an antecedent-consequent relationship in a script. Step 1110 in which the material synthesis device inputs, into a queue, feasible commands will be described in more detail below with reference to FIG. 12.
In step 1120, the material synthesis device may execute the commands included in the queue by hardware devices based on a monitoring result of the availability of the hardware devices. Step 1120 in which the material synthesis device executes commands included in a queue will be described in more detail below with reference to FIG. 13.

FIG. 12 is a flowchart illustrating an example of inputting, into a queue, commands in a feasible state according to an example embodiment. Referring to FIG. 12, the material synthesis device according to an example embodiment may input, into a queue, commands in a feasible state in step 1110 through steps 1210 to 1230 described below.
In step 1210, the material synthesis device may change, to a feasible state, a command state of commands whose preceding commands are in a completed state among commands that are in an idle state among commands included in a command list.
In step 1220, the material synthesis device may input, into a queue, the commands in the feasible state changed in step 1210. The queue may correspond to a space where commands to be executed by the dispatcher are stored. The dispatcher may check the queue to execute the commands included in the queue.
In step 1230, the material synthesis device may finish the execution of a script based on whether a command state of all the commands included in the queue in step 1220 is a completed state. When the command state of all the commands included in the queue is not the completed state, the material synthesis device may execute commands that remain in the queue without being executed.

FIG. 13 is a flowchart illustrating an example of executing commands included in a queue according to an example embodiment. Referring to FIG. 13, the material synthesis device according to an example embodiment may execute commands included in a queue in step 1120 through steps 1310 to 1360 described below.
In step 1310, the material synthesis device may score priorities corresponding to commands included in a queue and sort the commands in order of highest score.
In step 1320, the material synthesis device may execute a command using at least one hardware device that is in an idle state among hardware devices on which the commands sorted in step 1310 are to be executed.
In step 1330, the material synthesis device may generate a monitoring thread to monitor whether the execution of the command is successful.
In step 1340, the material synthesis device may determine whether the execution of the command is successful based on a monitoring result obtained in step 1330.
In step 1350, when the execution of the command is determined to be successful in step 1340, the material synthesis device may change a command state of the command to a completed state.
In step 1360, when the execution of the command is determined to be unsuccessful (i.e., failed) in step 1340, the material synthesis device may change the command state of the command to an aborted state.

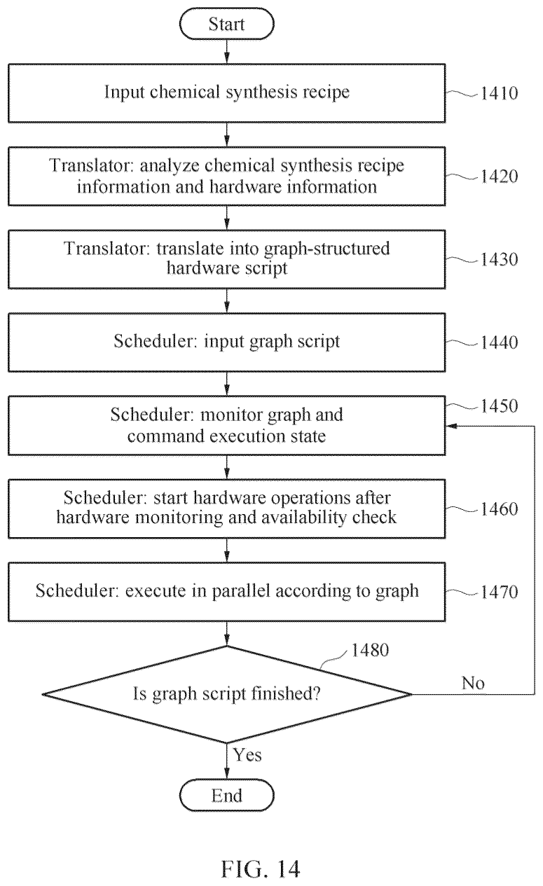

FIG. 14 is a flowchart illustrating a method of operating a material synthesis device according to an example embodiment. Referring to FIG. 14, the material synthesis device according to an example embodiment may translate an experiment recipe into a graph script and perform parallel scheduling in steps 1410 to 1480 described below.
In step 1410, the material synthesis device may receive, as an input, an experiment recipe. The experiment recipe may be, for example, but is not necessarily limited to, a chemical synthesis recipe.
In step 1420, the material synthesis device may analyze information about the experiment recipe and information about hardware devices by the recipe translator.
In step 1430, the material synthesis device may translate, by the recipe translator, the experiment recipe into a graph-structured hardware script based on an analysis result obtained in step 1420. Here, the graph-structured hardware script may correspond to a script in the form of a graph as described above, i.e., a graph script. The recipe translator may analyze the information about the experiment recipe, which includes chemical information, and the information about the hardware devices, which is hardware information including hardware configuration and constraints, and may then translate the experiment recipe into hardware commands. The recipe translator may translate the experiment recipe into the graph-structured hardware script by representing chemical synthesis processes corresponding to the experiment recipe as a combination of commands and deriving an antecedent-consequent relationship between the commands, based on the analyzed information.
In step 1440, the material synthesis device may input the script translated in step 1430 into the graph scheduler. The graph script generated as described above may be input to the graph scheduler, and an antecedent-consequent relationship of a graph in the graph script and command information may be stored in an internal memory of the graph scheduler.
In step 1450, the material synthesis device may monitor an execution state of the graph and the commands by the graph scheduler. The material synthesis device may monitor a current state of the graph and an execution state of the commands by a handler, which is one of the components of the graph scheduler.
In step 1460, the material synthesis device may, by the graph scheduler, monitor hardware devices and check the availability of the hardware devices identified based on a monitoring result to initiate operations by the hardware devices. The material synthesis device may check the availability of the hardware devices by a dispatcher, which is one of the components of the graph scheduler, to perform the operations by the hardware devices.
In step 1470, the material synthesis device may execute the commands in parallel according to a type of the graph by the graph scheduler. For example, when a command is in a feasible state on a corresponding hardware device, while monitoring the hardware devices by the dispatcher, the material synthesis device may transfer the command to the hardware device and perform an operation. According to a type of a script, multiple commands may be executed in parallel on specified hardware devices, and when the execution of the commands is completed, the graph script may be updated by the handler.
In step 1480, the material synthesis device may determine whether the graph script is completed. When it is determined in step 1480 that the graph script has not been completed, the material synthesis device may perform step 1450.

When it is determined in step 1480 that the graph script has been completed, the material synthesis device may end the steps.

The material synthesis device may repeat the process described above and end the steps when the execution of the graph script has been entirely completed.

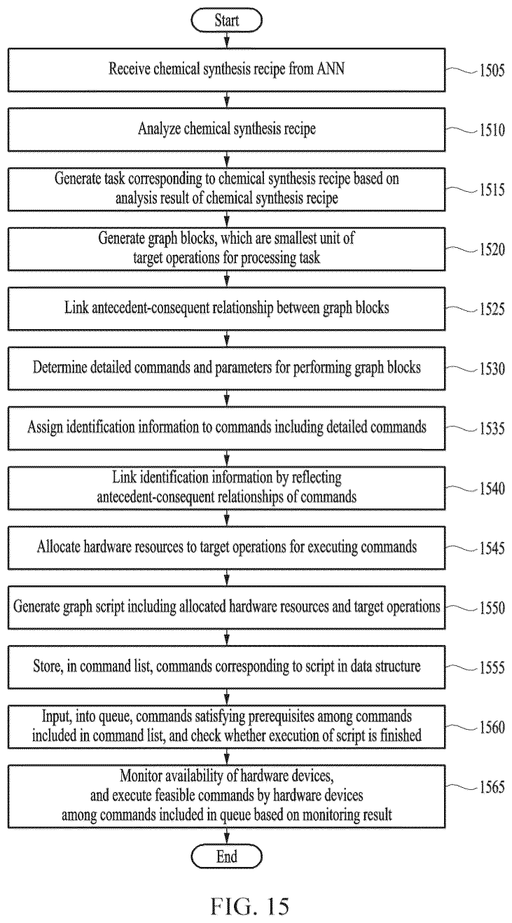

FIG. 15 is a flowchart illustrating a method of operating a material synthesis device according to an example embodiment. Referring to FIG. 15, the material synthesis device according to an example embodiment may execute commands in steps 1505 to 1565 described below.
In step 1505, the material synthesis device may receive a chemical synthesis recipe from an ANN.
In step 1510, the material synthesis device may analyze the chemical synthesis recipe received in step 1505.
In step 1515, the material synthesis device may generate a task corresponding to the chemical synthesis recipe based on an analysis result of the chemical synthesis recipe obtained in step 1510.
In step 1520, the material synthesis device may generate graph blocks, which are the smallest unit of target operations for processing the task generated in step 1515.
In step 1525, the material synthesis device may link an antecedent-consequent relationship between the graph blocks generated in step 1520.
In step 1530, the material synthesis device may determine detailed commands and parameters for performing the graph blocks whose antecedent-consequent relationship is linked in step 1525.
In step 1535, the material synthesis device may assign identification information to commands including the detailed commands.
In step 1540, the material synthesis device may link the identification information assigned in step 1535 by reflecting an antecedent-consequent relationship of the commands.
In step 1545, the material synthesis device may allocate hardware resources to the target operations for executing the commands linked to the identification information in step 1540.
In step 1550, the material synthesis device may generate a graph script including the hardware resources and target operations allocated in step 1545.
In step 1555, the material synthesis device may store, in a command list, commands corresponding to the script generated in step 1550 in a data structure.
In step 1560, the material synthesis device may input, into a queue, a command satisfying prerequisites among the commands included in the command list, by the handler of the graph scheduler, to check whether the execution of the script is completed.
In step 1565, the material synthesis device may, by the dispatcher of the graph scheduler, monitor the availability of the hardware devices and, based on a monitoring result, execute feasible commands among the commands included in the queue by the hardware devices. The material synthesis device may invoke a command by an API of the graph scheduler and execute the invoked command in the same form independently on at least one hardware device that is in an idle state.

FIG. 16 is a block diagram illustrating a material synthesis platform according to an example embodiment. Referring to FIG. 16, a material synthesis platform 1600 according to an example embodiment may include an ANN 1610, a material synthesis device 1630, and at least one hardware module 1650 including hardware devices.

The ANN 1610 may generate a chemical synthesis recipe. The ANN 1610 may correspond to a prediction model that is trained in advance to compute a synthesis method for producing a target product. The ANN 1610 may be at least one of, but is not necessarily limited to, a deep neural network (DNN), a recurrent neural network (RNN), and a conditional variational auto-encoder (CVAE).

The synthesis method may include, for example, synthesis pathways and synthesis conditions. A synthesis pathway may refer to a chemical reaction using reactants to produce a product. For example, in a case of generating a biaryl compound, which is a product, using an organoboron compound and a halogenated aryl compound, which are reactants, a synthesis pathway may be a Suzuki-Miyaura reaction. There may be multiple synthesis pathways depending on structural information of a reactant and structural information of a product.

A synthesis condition may refer to a set of conditions under which a chemical reaction using reactants to produce a product proceeds, and there may be at least one synthesis condition for a single synthesis pathway. The synthesis condition may include, for example, but is not necessarily limited to, at least one of a catalyst, a base, a solvent, a reagent, a temperature, and a reaction time.

The material synthesis platform 1600 may use the ANN 1610 (e.g., a synthesis prediction model) that is trained in advance to obtain a predicted synthesis result based on structural information of reactants, structural information of products, synthesis pathways, and synthesis conditions. For example, the predicted synthesis result may include, but is not necessarily limited to, at least one of a predicted yield, a predicted amount of a product, and a predicted synthesis time.

The material synthesis device 1630 may generate a script in the form of a graph representing an antecedent-consequent relationship between target operations corresponding to a chemical synthesis recipe, based on an analysis result of the chemical synthesis recipe. The material synthesis device 1630 may monitor the availability of hardware devices and, based on a monitoring result, schedule in parallel commands that satisfy an antecedent-consequent relationship in the script. The material synthesis device 1630 may be an operating system (OS) that links the ANN 1610 to the at least one hardware module 1650 to perform a material synthesis process. The material synthesis device 1630 may be, but is not necessarily limited to, the material synthesis device 100 described above.

The at least one hardware module 1650 may include hardware devices that perform target operations according to the commands scheduled in parallel by the material synthesis device 1630.

Each of the at least one hardware module 1650 may have independent functionality. For example, one hardware module 1650 may include multiple types of hardware devices.

For example, in a case where the at least one hardware module 1650 is a dispensing module that performs automated dispensing of reagents, the dispensing module may include various hardware devices, such as, for example, a powder dispenser, a solvent dispenser, a liquid dispenser, a vial loader, a robot, and a capper described above with reference to FIG. 5.

The unit of operations in the material synthesis platform 1600 may be a recipe (e.g., the chemical synthesis recipe). For example, in a case where one recipe is processed at a time in one hardware module, the processing in the hardware module may be represented sequentially or serially, and scheduling in the hardware module may be relatively simply operated.

The material synthesis platform 1600 according to an example embodiment may also process multiple recipes simultaneously by appropriately allocating resources (e.g., hardware devices) of multiple hardware modules through parallel scheduling. The material synthesis platform 1600 may perform parallel processing by the hardware devices in consideration of the characteristics of the hardware devices in the multiple hardware modules and antecedent-consequent relationships of operations.

The example embodiments described herein may be implemented using hardware components, software components and/or combinations thereof. A processing device may be implemented using one or more general-purpose or special purpose computers, such as, for example, a processor, a controller, an arithmetic logic unit (ALU), a digital signal processor, a microcomputer, a field programmable gate array (FPGA), a programmable logic unit (PLU), a microprocessor, or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art may appreciate that a processing device may include multiple processing elements and multiple types of processing elements. For example, a processing device may include multiple processors or a processor and a controller. In addition, different processing configurations may be possible, such as, parallel processors.

The software may include a computer program, a piece of code, an instruction, or some combination thereof, to independently or collectively instruct and/or configure the processing device to operate as desired. The software and/or data may be embodied permanently or temporarily in any type of machine, component, physical or virtual equipment, computer storage medium or device, or in a propagated signal wave capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described examples may be recorded in non-transitory computer-readable media including program instructions to implement various operations of the above-described examples. The media may also include, alone or in combination with the program instructions, data files, data structures, and the like. The program instructions recorded on the media may be specially designed and constructed for the purposes of examples, or they may be of the kind well-known and available to those having skill in the computer software arts. Examples of non-transitory computer-readable media include magnetic media such as hard disks, floppy disks, and magnetic tape; optical media such as compact disc read-only memory (CD-ROM) discs, digital versatile discs (DVDs), and/or Blue-ray discs; magneto-optical media such as optical discs; and hardware devices that are specially configured to store and perform program instructions, such as ROM, RAM, flash memory (e.g., universal serial bus (USB) flash drives, memory cards, memory sticks, etc.), and the like. Examples of program instructions include both machine code, such as produced by a compiler, and files containing higher-level code that may be executed by the computer using an interpreter.

The above-described hardware devices may be configured to act as one or more software modules in order to perform the operations of the above-described examples, or vice versa.

While the present disclosure includes specific examples, it is to be apparent after an understanding of the present disclosure that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, in addition to the above disclosure, the scope of the disclosure may also be defined by the claims and their equivalents, and all variations within the scope of the claims and their equivalents are to be construed as being included in the disclosure.

What is claimed is:

1. A material synthesis device, comprising:
at least one processor configured to be implemented as:
a recipe translator configured to, based on analyzing a chemical synthesis recipe, generate a script in the form of a graph representing an antecedent-consequent relationship between target operations corresponding to the chemical synthesis recipe; and
a graph scheduler configured to monitor availability of hardware devices of a material synthesis platform, and schedule and execute in parallel commands satisfying the antecedent-consequent relationship in the script based on the monitoring.

2. The material synthesis device of claim 1, wherein the recipe translator is further configured to:
generate the script in the form of the graph representing commands configured to perform the target operations in the hardware devices, based on the analyzing of the chemical synthesis recipe and at least one of information with respect to the hardware devices.

3. The material synthesis device of claim 2, wherein the information with respect to the hardware devices comprises at least one of:
a database corresponding to the hardware devices, configuration information of the hardware devices, the antecedent-consequent relationship between the target operations performed on the hardware devices, a movement flow of a robot configured to perform the target operations with respect to a moving target, code corresponding to the hardware devices, and a description language of the hardware devices.

4. The material synthesis device of claim 1, wherein the recipe translator is further configured to:
obtain the antecedent-consequent relationship between the target operations based on the chemical synthesis recipe, based on configuration information of the hardware devices.

5. The material synthesis device of claim 1, wherein the recipe translator is further configured to:
generate graph blocks, which are respectively a unit of performing the target operations; and
structure a synthesis process based on the chemical synthesis recipe by the graph blocks to represent the chemical synthesis recipe as the script.

6. The material synthesis device of claim 1, wherein the script comprises:
node information comprising information with respect to the hardware devices configured to perform the target operations, detailed information with respect to the target operations, priorities of the target operations based on an importance of the target operations, and a command state configured to perform the target operations; and
edge information corresponding to an antecedent-consequent relationship between a preceding node and a following node included in graph blocks.

7. The material synthesis device of claim 6, wherein the command state comprises at least one of:
an idle state, a feasible state, a processing state, a completed state, and an aborted state.

8. The material synthesis device of claim 1, wherein the graph scheduler comprises:
a handler configured to generate and manage a command list comprising commands satisfying the antecedent-consequent relationship, based on whether execution of preceding nodes is completed in the script;
a dispatcher configured to monitor the availability of the hardware devices, and execute at least some of the commands included in the command list by the hardware devices based on the monitoring; and
a controller configured to control operations of the handler and the dispatcher, based on a user command from a user.

9. The material synthesis device of claim 8, wherein the handler is configured to:
input, into a queue, commands having a command state that is a feasible state among the commands included in the command list; and
determine whether the script is completed.

10. The material synthesis device of claim 9, wherein the dispatcher is configured to:
score priorities of the commands included in the queue and sort the commands included in the queue in order of highest score;
execute the sorted commands based on at least one hardware device that is in an idle state among the hardware devices configured to execute the sorted commands; and
change a command state of the sorted commands based on monitoring whether execution of the sorted commands is successful.

11. The material synthesis device of claim 1, wherein the hardware devices are configured to:
perform the target operations based on the parallel scheduled script.

12. The material synthesis device of claim 1, wherein the chemical synthesis recipe and the script are in a JavaScript Object Notation (JSON) file format.

13. A material synthesis platform, comprising:
an artificial neural network (ANN) configured to generate a chemical synthesis recipe;
a material synthesis device at least one processor configured to:
generate, based on analyzing the chemical synthesis recipe, a script in the form of a graph representing an antecedent-consequent relationship between target operations corresponding to the chemical synthesis recipe;
monitor availability of hardware devices; and
schedule, in parallel, commands satisfying the antecedent-consequent relationship in the script based on a result of the monitoring; and
at least one hardware module comprising the hardware devices configured to perform the target operations based on the commands scheduled in parallel.

14. A method of operating a material synthesis device, the method comprising:
analyzing a chemical synthesis recipe;
generating, based on analyzing the chemical synthesis recipe, a script in the form of a graph representing an antecedent-consequent relationship between target operations corresponding to the chemical synthesis recipe;
monitoring availability of hardware devices of a material synthesis platform; and
scheduling and executing, in parallel, commands satisfying an antecedent-consequent relationship in the script based on the monitoring.

15. The method of claim 14, wherein the generating of the script comprises:
structuring a synthesis process based on the chemical synthesis recipe by graph blocks, which are a smallest unit of the target operations configured to process a task corresponding to the chemical synthesis recipe, based on the analyzing of the chemical synthesis recipe;
allocating hardware resources to the target operations configured to execute the commands configured to perform the graph blocks based on a result of the structuring; and
generating the script comprising the allocated hardware resources and the target operations.

16. The method of claim 15, wherein the structuring of the synthesis process comprises:
linking an antecedent-consequent relationship between the graph blocks;
determining at least one of detailed commands and parameters configured to perform the graph blocks;
assigning identification information to commands comprising the detailed commands; and
linking the identification information by reflecting an antecedent-consequent relationship of the commands to structure the synthesis process based on the chemical synthesis recipe.

17. The method of claim 15, wherein the generating of the script comprises:
obtaining the antecedent-consequent relationship between the target operations that execute in parallel a synthesis based on the chemical synthesis recipe, based on a result of analyzing the chemical synthesis recipe and at least one of information about the hardware devices; and
generating the script in the form of the graph representing commands for the hardware devices, based on the antecedent-consequent relationship between the target operations.

18. The method of claim 14, wherein the scheduling and executing, in parallel, of the commands satisfying the antecedent-consequent relationship in the script comprises:
inputting, into a queue, a command that is in a feasible state among commands stored in a command list comprising the commands satisfying the antecedent-consequent relationship in the script; and
executing commands included in the queue by the hardware devices based on the monitoring of the availability of the hardware devices.

19. The method of claim 18, wherein the inputting of the command in the feasible state into the queue comprises:
changing, to a feasible state, a command state of commands whose preceding commands are in a completed state among commands whose command state is an idle state among the commands included in the command list; and
inputting, into the queue, the commands whose command state are changed to the feasible state.

20. The method of claim 18, wherein the executing of the commands included in the queue comprises:
scoring priorities corresponding to the commands included in the queue and sorting the commands in order of highest score;
executing the command based on at least one hardware device that is in an idle state among hardware devices configured to execute the sorted commands;
generating a monitoring thread and monitoring whether execution of the command is successful;
based on the execution of the command being monitored as being successful, changing a command state of the command to a completed state; and
based on the execution of the command being monitored as being unsuccessful, changing the command state of the command to an aborted state.
